(19) United States
(12) Patent Application Publication
(10) Pub. No.: US 2008/0254201 A1
(43) Pub. Date: Oct. 16, 2008

(54) Method for Preparing Endosseous Implants with Zircon Dioxide Coating

(75) Inventors: Francesco Carinci, Bologna (IT); Carlo Alberto Bignozzi, Ferrara (IT); Claudio Minero, Racconigi (IT); Valter Maurino, Pinerolo (IT); Stefano Caramori, Viconovo (IT)

Correspondence Address: WILLIAM J. SAPONE;COLEMAN SUDOL SAPONE P.C., 714 COLORADO AVENUE, BRIDGE PORT, CT, 06605, US

(73) Assignee: GUYA BIOSCIENCE S.R.L., Ferrara (IT)

(21) Appl. No.: 11/577,517

(22) PCT Filed: Oct. 13, 2005
(86) PCT No.: PCT/IB2005/003162

§ 371 Date: Jan. 30, 2008

(30) Foreign Application Priority Data
Oct. 22, 2004 (IT) BO2004A 000654

(51) Int. Cl. A61L 27/30

(52) U.S. Cl. 427/2.27

(57) ABSTRACT

The method includes the following steps: formulation of liquid, non-gelled and stable precursors by solvolysis of ZV (IV) compounds; precursor deposition on endosseous implant surface; thermal treatment to achieve film densification, in the presence of oxygen, of a complex formed by the said endosseous implant and precursor, to obtain on the implant surface a thin nanocrystalline zirconium dioxide film.

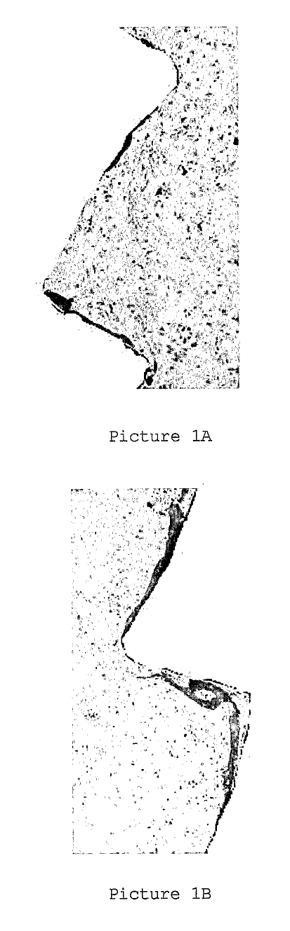

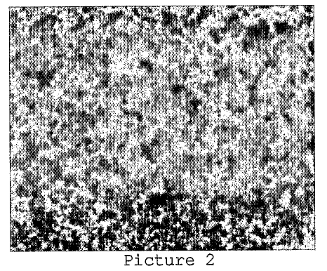

### TECHNICAL FIELD OF THE INVENTION

The invention relates to the technical field concerning the preparation of endosseous implants with high osseointegration degree, and in particular the invention concerns a method for preparing a fully-anchored zirconium dioxide film with non-gelled organic doped precursors on endosseous implants.

### PRIOR ART

It is well known that zirconium is widely used for medical purposes for its mechanical properties and its biocompatibility.

Biological compatibility can be detected not only in the absence of inflammatory rejection crisis, but also in the increase of biological process of the receiving tissue, in the case of endosseous prosthesis is expressed in an increased osseointegration.

Zirconium is extensively used in medical purposes thanks to its mechanical properties and biocompatibility. From ninety, zirconium biocompatibility has been demonstrated, in several scientific report concerning in vitro studies, in vivo models and clinical trials.

Concerning dental implants used as root of lost teeth substitutes and in the orthopedics reconstructive surgeon, zirconium biocompatibility is show in its capability to determine osseointegration. Osseointegration of a fixture in bone is defined as the close apposition of new and reformed bone in congruence with the fixture.

When process is completed a direct, structural and functional, connection is established, capable of carrying normal physiological loads without excessive deformation and without initiating rejecting mechanisms. The processes intervene in a lapse of time of sixty days, comparable to the fracture fixing process. This period can differ occurring others variables such as: microgap dimension among implant and bone, primary implant stability, type of implant surface, etc.

Osseointegration may depend on some specific implant features: a) type of material, b) macroscopic surface design (i.e. screw design in root-form dental implants), c) type of surface. Factor c) is determined according to the manufacturing technique adopted, for example smooth or rough. The surface is important for creation of implant surface microroughness is needed for filopodi osteoblast anchorage. However factor a) is the most important to determine osseointegration. In fact during sixties iron made implants were used. Branemark together with other scientists (Branemark P I, Adell R, Breine U, Hansson B O, Lindstrom J, Ohlsson A. “Intra-osseous anchorage of dental prostheses. I. Experimental studies.” Scand. J. Plast. Reconstr. Surg. 1969; 3(2):81-100. Adell R, Hansson BO, Branemark P I, Breine U. “Intra-osseous anchorage of dental prostheses. II. Review of clinical approaches.” Scand. J. Plast. Reconstr. Surg. 1970; 4(1):19-34) demonstrated with their studies that, differently from iron, titanium is capable of stimulate osteogenesis. Since then all endosseous implants are made of titanium. Since nineties, nevertheless, several studies has been carried out on zirconium, not only used to manufacture crowns but also as potential root substitute, due to its ivory natural root color likeness (Scarano A, Di Carlo F, Quaranta M, Piattelli A. “Bone response to zirconia ceramic implants: an experimental study in rabbits.” J. Oral Implantol. 2003; 29(1):8-12 and bibliography quoted in the article).

### DESCRIPTION OF THE INVENTION

The object of the disclosed invention is to propose a coating method on metallic supports with thin zirconium dioxide nanocrystalline film, fostering osseointegration. Another object of the present invention is to propose a coating method to obtain a high osseointegration degree of endosseous implants.

A further object of the present invention is to propose a coating method using a stable and compact film on the surface of the treated implant.

Moreover it must be added the intention to propose a method strengthened by simple and quick phases.

The above mentioned objects are obtained in accordance with the contents of claims, by a method for preparing a fully-anchored zirconium dioxide film with non-gelled organic doped precursors on endosseous implants, including the following steps:

formulation of liquid, non-gelled and stable precursors by solvolysis of Ti(IV) compounds;

precursor deposition on endosseous implant surface;

thermal treatment to achieve film densification, in the presence of oxygen, of a complex formed by the above mentioned endosseous implant and precursor, to obtain on the implant surface a thin nanocrystalline zirconium dioxide film.

### BRIEF DESCRIPTION OF THE PICTURES

The characteristics features are pointed out in the following with particular reference to the enclosed pictures, in which:

picture 1A shows an example of osteogenesis stimulation on uncoated surface;

picture 1B shows an example of osteogenesis stimulation on zirconium dioxide fully coated surface, made according to the method describe in this invention;

picture 2 shows an electronic microscope zoom on zirconium dioxide coated surface.

### PREFERRED EMBODIMENTS OF THE INVENTION

The process of the disclosed invention is defined in several steps, and concerns the formulation of liquid, non-gelled colloidal nanocrystalline precursors based on zirconium dioxide.

Precursor can easily deposited on the dental implant surface, using simple techniques for immersion and extraction at a controlled speed (i.e. dip-coating process), followed by a thermal treatment to achieve film densification.

The method set in this invention consist of the deposition on the metallic dental implant support or endosseous implants in general, of a stable liquid precursor made of inorganic compounds of Zirconium(IV), partially or totally hydrolyzed, and a suitable organic doping, in particular s-triazine derivates, included to improve the biocompatibility and mechanical resistance.

Then the endosseous implant is undergone to a thermal treatment to achieve film densification.

Liquid precursor is made of inorganic compounds of Zirconium(IV), partially or totally hydrolyzed, in which gelation is avoided.

The formulations of non-gelled liquid precursors comprise Zirconium (IV) at concentrations in the range 0.10 to 40% by weight.

The above mentioned compounds contain in their formulations tetraisopropoxy zirconium and tetrabutoxy zirconium.

Solvolysis of Ti(IV) compounds needs from 1 minute to 36 hours, at temperatures ranging from 5° C. to the solvent boiling point, eventually under pressure (1-20 atm) at temperatures ranging from 0° C. to 120° C.

The solvolysis is necessary to form compounds of Zr(IV) that are less volatile than the original compounds, unable to vaporize during the subsequent thermal treatment, and showing good film sticking properties, and sufficient thickness to the support.

Otherwise, the precursor could be partially or completely vaporized and lost during the thermal treatment, with formation of irregular and/or discontinuous or no coatings.

The water concentration needed by the hydrolysis ranges from 0.1 to 30% by weight.

Organic solvents, which are alcohols, also polyfunctional and containing oxygen in ether bonds, carry 1-10 carbon atoms and 1-6 oxygen atoms, or lactones containing 4-6 carbon atoms, or mixtures thereof in all proportions.

The solvent choice is made according to procedures used for deposition (dip-coating, spray or roll-coating) and the Zirconium dioxide film layer thickness desiderated.

An example about film morphologic features obtained by means of spray coating is shown in picture 3.

It shows a 4 nm deposition of ZrO₂, with an average distribution of 20 nm diameter nanoparticles.

The gelation of the liquid precursor, either contemporary to the preparation step or when the precursor is stored before deposition renders it incompatible with the deposition with dip-coating, spray or roll-coating, especially if thin films below 10 μm are desired.

To avoid gelation an inorganic or organic acid is added at concentrations ranging from 0.1% to 20% by weight and/or a surfactant of type nonionic, or cationic, or anionic, or zwitterionic and their mixtures in all proportions, at concentrations ranging from 1% to 20% by weight.

The presence of surfactant and/or the acid as the additional effect of inhibiting the formation in the liquid precursor of zirconium dioxide particles exceeding the critical diameter threshold estimated in 100 nm: beyond this dimension particles will form coating films less resistant to the abrasion and less uniform.

The gelation processes and formation of particulate titanium dioxide are inhibited by the presence of the acid and/or the surfactant at temperatures ranging from −10° C. to 120° C.

At ambient temperature (not higher than 30° C.) the disclosed formulation renders the precursor stable against gelation and particle formation and settling for 6 to 12 months, depending on the composition.

Among inorganic acids the following are suitable: nitric acid, sulfuric acid, phosphoric acid, hydrofluoric acid, hydrochloric acid, perchloric acid and their mixtures in all proportions.

Among organic acids are adequate those with linear or branched chains, also with 2 or 3 carboxylic groups and/or containing hydroxyl-, and/or chloro-, and/or fluoro-, and/or bromo-groups, or benzoic acid and its derivates, and/or other carboxylic acid with aromatic structure.

Non-ionic surfactants alkyl- or alkylarylethoxilate and their mixtures in all proportions (for example the commercial products Brij 30, Brij 35, Triton X100), and/or alkyl or alkylethoxysulphate anionic surfactants (for example sodium dodecyl sulphate), and/or alkylbenzene sulphonate, and/or cationic surfactants, e.g. cetyltrimethylammonium bromide, and/or zwitterionic surfactants, like betaine derivates, are among the surfactants useful to block the gelation and ZrO2 particles growth.

S-triazine ring is characterized by compounds like melamine, acid cyanuric, cyanurate chloride, and it has been discovered to have positive effect on quality deposition.

Various s-triazine compounds show an excellent thermal stability (see for example E. M. Smolin, L. Rapoport, s-Triazines, in: A. Weissenberg Ed., The Chemistry of Heterocyclic Compounds, Vol. 13°, Wiley Interscience, New York, 1959).

S-triazine doping confers to the film an excellent stability and a very good adhesion to metallic support. The coating with the precursor made by the above cited procedures, is followed by a thermal treatment lasting 10-200 min at temperatures ranging from 300° C. to 800° C., in the presence of a gas phase containing oxygen in the range 1% to 50% by volume, in order to fully convert the precursor in microcrystalline anatase TiO2, and obtain a coating with good mechanical and chemical stability.

Table 1 shows the best way to implement the invention by one example of composition of the precursor used for s-triazine coating of endosseous implants, like cyanuric acid, type of support used and temperatures of thermal treatment.

Sometimes the preparation has to be performed under nitrogen atmosphere, depending on the organic solvent.

A thermal aging could improve precursors performance completing hydrolysis and/or solvolysis of the original zirconium compound. After thermal aging, the exemplified formulation neither gel nor form solid core particles and can be stored for 6-12 months, at room temperature (less than 25° C.) in a sealed case.

TABLE 1

| | | |
|---|---|---|
| | | Preferred |
| Component | Weight % | Weight % |

Step 1: Liquid precursor formulation

| | | |
|---|---|---|
| Isopropyl alcohol | 36 to 87.7 | 72 |
| Phosphoric acid 85% | 0.1 to 10 | 0.3 |
| Water | 0.1 to 5 | 2.8 |
| Triton X 100 | 1 to 20 | 2.1 |
| Zirconium(IV) | 10 to 30 | 17.4 |
| compound | | |
| tetraisopropoxy- | | |
| isopropanol | | |
| Zr[OCH(CH₃)₂]₄(CH₃)₂CHOH | | |
| Cyanurate chloride | 1 to 20 | 6.5 |

Step 2: Solvolysis: closed container from 24 h at 60° C.
Step 3: Coating and film densification

| | |
|---|---|
| Deposition procedure | Dip-coating, rate 12 cm min⁻¹ |
| Support | Titanium or other metallic material |
| Thermal treatment | 700° C. for 60 min under forced air |
| | flow |

Example of titanium dental endosseous implants coated with zirconium dioxide film s-triazine doped according to the herein disclosed procedure. It is shown the weight % of initial Zr(IV) compound, acid, surfactant and solvent, the coating procedure and details about thermal treatment.

A biological test performed on animal model consist in bone grafts two implant series (coated and uncoated) in rabbit tibia. Animals are sacrificed after 30 days and the block section, containing the implant, is retrieved for histomorfometric analysis evaluation. The golden standard for the biological test consisting in bone grafts alloplastic material in rabbit femur/tibia, is the system internationally recognized for biocompatibility trials (Scarano A, Di Carlo F, Quaranta M, Piattelli A. “Bone response to zirconia ceramic implants: an experimental study in rabbits.” J. Oral Implantol. 2003; 29(1):8-12. Piattelli M, Scarano A, Paolantonio M, Iezzi G, Petrone G, Piattelli A. “Bone response to machined and resorbable blast material titanium implants: an experimental study in rabbits.” J. Oral Implantol. 2002; 28(1):2-8. Cordioli G, Majzoub Z, Piattelli A, Scarano A. “Removal torque and histomorphometric investigation of 4 different titanium surfaces: an experimental study in the rabbit tibia.” Int. J. Oral Maxillofac. Implants. 2000 September-October; 15(5):668-74. Piattelli A, Scarano A, Di Alberti L, Piattelli M. “Histological and histochemical analyses of acid and alkaline phosphatases around hydroxyapatite-coated implants: a time course study in rabbit”, Biomaterials. 1997 September; 18(17):1191-4).

From experimental data, as shown in histological pictures 1 and 3 at 30 days, coated surface demonstrated a clear neosteogenesis stimulation. Picture 3 shows a 43% increasing of bone tissue if compared to uncoated specimen in picture 1, improve osteogenesis of the zirconium dioxide coated surface.

Coatings made with the disclosed method form thin film of 0.05-10 μm thickness, resistant to atmospheric factors, abrasions and completely homogeneous. The deposition process can be applied to a large number of dental implants, or endosseous implants in general, applied on a proper material support allowing film deposition, for instance by means of immersion and extraction of the support at a controlled speed. Following thermal treatment of coated implants allow deposition of a nanocrystalline zirconium dioxide coating film, showing the following strengths: improves osseointegration; allows to manufacture endosseous implants with zirconium dioxide coated supports of materials different from titanium (i.e. iron).

Surface coated following the disclosed method is capable of improving new bone apposition, which represents a key factor in the definition of prosthesis biocompatibility. The disclosed invention concerns formulations and processes capable of obtaining an improved osseointegration of dental implants. The claimed procedure allows avoiding the gelification of the precursor, running away the need of further repeptization of the gel as usually required in common solgel methods. The liquid precursor is stable in air, and storable for some months without alteration.

The disclosed invention concerns the formulation of liquid, non-gelled and stable precursors for a low cost manufacturing coating film process (dip-coating, spray or roll-coating).

The film obtained according to the procedure herein shows very good mechanical properties, adhesion to the metallic support and abrasion resistance without the intervention of an in-between layer.

It is understood that what above, has been described as a pure, not limiting example, therefore, possible practical-applications variants of the proposed steps remain within the protective scope of the invention, as described above and claimed hereinafter.

1. Method for preparing a fully-anchored zirconium dioxide film with non-gelled organic doped precursors on endosseous implants, characterized in that it includes the following steps:
formulation of liquid, non-gelled and stable precursors by solvolysis of Ti(IV) compounds;
precursor deposition on endosseous implant surface;
thermal treatment to achieve film densification, in the presence of oxygen, of a complex formed by the said endosseous implant and precursor, to obtain on the implant surface a thin nanocrystalline zirconium dioxide film.

2. Method, according to claim 1, characterized in that said liquid non-gelled precursor includes:
a Zirconium (IV) compound at concentrations as Zirconium dioxide equivalent, in the range 10% to 30% by weight of the liquid precursor;
water at concentrations in the range 0.1% to 5% by weight;
an organic solvent;
an organic or mineral acid and their mixtures, at concentrations in the range 0.1% to 20%, avoiding the gelification of the precursor;
a surfactant of type nonionic, or cationic, or anionic, or zwitterionic and their mixtures in all proportions, at concentrations ranging from 0.1% to 20% by weight.

3. Method, according to claim 1, characterized in that said film entirely coats endosseous implant surface.

4. Method, according to claim 1, characterized in that said solvolysis of Zr(IV) compounds needs from 1 minute to 36 hours.

5. Method, according to claim 3, characterized in that said solvolysis is performed at concentrations ranging from 0° C. and solvent boiling point.

6. Method, according to claim 1, characterized in that said solvolysis of Zr(IV) compounds needs from 1 minute to 36 hours and it is performed at a temperature between 0° C. and the solvent boiling point.

7. Method, according to claims 1, characterized in that said solvolysis of Zr(IV) is performed at temperatures ranging from 0° C. to 120° C., under pressure (1-20 atm).

8. Method, according to claim 1, characterized in that said precursor deposition is performed by means of coating procedures such as dip-coating, spray-coating or roll coating.

9. Method, according to claim 1, characterized in that said presence of oxygen during the thermal treatment is in the range 1% to 50% by volume.

10. Method, according to claim 1, characterized in that said thermal treatment is performed at temperatures ranging from 300° C. to 800° C.

11. Method, according to claim 1, characterized in that said thermal treatment is performed at temperatures ranging from 300° C. to 800° C., in the presence of a gas phase containing oxygen in the range 1% to 50% by volume.

12. Method, according to claims 2, characterized in that said compounds contain in their formulation tetrabutoxy.

13. Method, according to claims 2, characterized in that said compounds contain in their formulation tetraisopropoxy-isopropanol.

14. Method, according to claim 2, characterized in that said organic solvent, includes an alcohol, polyfunctional and containing oxygen in ether bonds, carrying 1-10 carbon atoms.

15. Method, according to claim 1, characterized in that said phase of liquid precursors deposition followed by a thermal treatment is repeated a predetermined number of times.

16. Method, according to claim 2, characterized in that said precursors include one transitional element at least.

17. Method, according to claim 2, characterized in that said precursors include one transitional element belonging to group IVA, in an atomic proportion with Zr(IV) up to 25%.

18. Method, according to claim 2, characterized in that said solvolysis of Zr(IV) compounds needs from 1 minute to 36 hours and it is performed at a temperature between 0° C. and the solvent boiling point.

19. Method, according to claim 5, characterized in that said solvolysis of Zr(IV) compounds needs from 1 minute to 36 hours and it is performed at a temperature between 0° C. and the solvent boiling point.

20. Method, according to claim 3, characterized in that said precursors include one transitional element at least.

21. Method, according to claim 3, characterized in that said precursors include one transitional element belonging to group IVA, in an atomic proportion with Zr(IV) up to 25%.
